(19) United States
(12) Patent Application Publication
(10) Pub. No.: US 2020/0038432 A1
(43) Pub. Date: Feb. 6, 2020

(54) HYALURONIC ACID COATED CHIMERIC VIRAL/NONVIRAL NANOPARTICLES

(71) Applicant: The Regents of the University of California, Oakland, CA (US)

(72) Inventors: Young Jik Kwon, Irvine, CA (US); Margaret Lugin, Irvine, CA (US)

(21) Appl. No.: 16/528,572

(22) Filed: Jul. 31, 2019

Related U.S. Application Data
(60) Provisional application No. 62/712,962, filed on Jul. 31, 2018.

(51) Int. Cl. A61K 31/728; A61K 9/14; A61K 48/00; C12N 15/86; C12N 7/00; A61K 31/7105

(52) U.S. Cl. CPC A61K 31/728; A61K 9/141; A61K 48/0041; B82Y 5/00; C12N 7/00; A61K 31/7105; C12N 15/86

(57) ABSTRACT

The disclosure provides for hyaluronic acid functionalized chimeric viral/nonviral nanoparticles, and uses thereof.

### CROSS REFERENCE TO RELATED APPLICATIONS

This application claims priority under 35 U.S.C. § 119 from Provisional Application Ser. No. 62/712,962 filed Jul. 31, 2018, the disclosure of which is incorporated herein by reference.

### STATEMENT OF GOVERNMENT SUPPORT

This invention was made with government support under Grant No. 1R21CA228099-01A1 awarded by the National Cancer Institute, and Grant No. 5T32AI7319-28 awarded by the National Institutes of Health. The government has certain rights in the invention.

### TECHNICAL FIELD

The disclosure provides for hyaluronic acid coated viral/nonviral nanoparticles, and uses thereof.

### BACKGROUND

Development of efficient and safe delivery methods remains a pivotal challenge in gene therapy. Recombinant viral vectors are superior to nonviral vectors in delivering genes, especially in vivo. Despite these advantages, viral vectors have some notable drawbacks, including eliciting an immune response in a host (particularly upon repeated administrations); are difficult to produced in large-scale; limitations in the size of genes that can be packaged; narrow cell tropisms; and lack of surface modalities for molecular (synthetic) modifications without altering viral stability and infectivity. Researchers have attempted to overcome some of the foregoing drawbacks. For example, immunosuppression has been used to prevent a host's immune response to the viral vector. Immunosuppression however increases the host chance's of coming down with an opportunistic infection. While genetically modifying the viral capsid and envelope, conjugating various functional moieties (e.g., targeting molecules), and electrostatically or covalently incorporating lipids or polymers are often accompanied by compromised infectivity or retained/new immunogenicity upon repeated administrations. Nonviral vectors using synthetic materials (e.g., cationic lipids and polymers), on the other hand, are easy to manufacture in a large scale, can deliver larger payloads, are readily tunable for desirable structure/performance, and exhibit low immunogenicity. Nevertheless, poor transfection efficiencies, particularly in vivo, has limited the use of nonviral vectors for gene therapy.

### SUMMARY

Intravitreal delivery of viral gene therapy for retinal diseases has been found to be promising as retinal cells are immune privileged and terminally differentiated. Accordingly, the use of viral gene therapy is expected to have more permanent results in treating retinal diseases, like age-related macular degeneration (AMD), than nonviral gene therapy. Hyaluronic acid (HA), a naturally occurring polysaccharide in the human body, has been found to improve the uptake of nonviral particles by retinal cells. Testing the effects of HA on ChNPs, it was found that ChNPs which were functionalized with HA, more efficiently transduced ARPE-19, a retinal cell line, than ChNPs without HA. In in vivo studies, it was further found that ChNPs which were functionalized with HA, preferentially localized in inner retinal cells, which was not the case with ChNPs not similarly functionalized. Further, HA functionalized ChNPs were also found to have greater efficacy than ChNPs without HA. Accordingly, the HA functionalized ChNPs of the disclosure provides for more efficient gene expression in retinal cells than other nonviral systems or to ChNPs that are not functionalized by HA.

In a particular embodiment, the disclosure provides a hyaluronic acid functionalized chimeric viral/nonviral nanoparticle comprising: (i) a core comprising a recombinant adeno-associated virus (AAV) that expresses a transgene; (ii) one or more acid labile degradable polymer layers surrounding the core that may further comprise encapsulated nucleic acids, CRISPR-Cas or CRISPRi systems, therapeutic proteins, or therapeutic drugs, wherein the acid degradable polymer layers hydrolyze in a mildly acidic environment; and (iii) an outer coating that is in contact with the one or more acid labile degradable polymer layers that is comprised of hyaluronic acid. In another embodiment, the recombinant AAV is AVV serotype 1, AVV serotype 2, AVV serotype 3, AVV serotype 5, AVV serotype 7, AVV serotype 8 or AVV serotype 9. In a particular embodiment, the AAV is AVV serotype 2 or AVV serotype 8. In a certain embodiment, the core comprises a recombinant AAV that expresses a gene therapy product from a transgene to treat a disease or disorder. In a further embodiment, the core comprises a recombinant AAV that expresses a gene therapy product from a transgene comprising a RPE65 gene, RPE65 gene, a Rab escort protein-1 (REP) gene, a retinoschisin (RS1) gene, a ciliary neurotrophic factor (CNTF) gene and/or a pigment epithelium-derived factor (PEDF) gene. In yet a further embodiment, the one or more acid labile degradable polymer layers are polyketal-based polymer layers. In another embodiment, the polyketal-based polymer layers are made from photo-polymerization of acid-cleavable amino ketal monomers having the structure of:

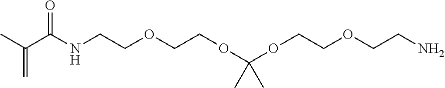 [SMILES: C=C(C)C(=O)NCCOCCOC(C)(C)OCCOCCN]

and acid-cleavable cross-linkers having the structure of:

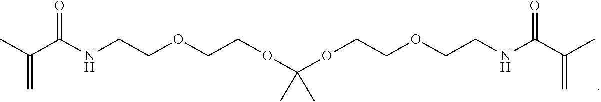 [SMILES: C=C(C)C(=O)NCCOCCOC(C)(C)OCCOCCNC(=O)C(=C)C]

In yet another embodiment, wherein eosin is used as a photoinitiator for the photo-polymerization of the acid-cleavable amino ketal monomers and acid-cleavable cross-linkers. In a certain embodiment, the hyaluronic acid functionalized chimeric viral/nonviral nanoparticle has a diameter of 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, or a range that includes or is between any two of the foregoing values, including fractional increments thereof. In a further embodiment, the hyaluronic acid functionalized chimeric viral/nonviral nanoparticle has a diameter from 100 nm to 1000 nm. In another embodiment, where in comparison to a chimeric viral/nonviral nanoparticle not functionalized with hyaluronic acid, the hyaluronic acid functionalized chimeric viral/nonviral nanoparticle exhibits less toxicity and/or improved localization in inner retinal cells. In yet another embodiment, the hyaluronic acid functionalized chimeric viral/nonviral nanoparticle has a zeta potential of 0 mV, −1 mV, −2 mV, −3 mV, −4 mV, −5 mV, −6 mV, −7 mV, −8 mV, −9 mV, −10 mV, −11 mV, −12 mV, −13 mV, −14 mV, −15 mV, −16 mV, −17 mV, −18 mV, −19 mV, −20 mV, −21 mV, −22 mV, −23 mV, −24 mV, −25 mV, −26 mV, −27 mV, −28 mV, −29 mV, −30 mV, or a range that includes or is between any two of the foregoing values, including fractional increments thereof. In a further embodiment, the hyaluronic acid functionalized chimeric viral/nonviral nanoparticle has a zeta potential from 0 mV to −30 mV. In another embodiment, the one or more acid labile degradable polymer layers surrounding the core comprise encapsulated gene silencing/editing oligonucleotides. In a further embodiment, the gene silencing/editing oligonucleotides are siRNA, miRNA or shRNA. In yet a further embodiment, the gene silencing/editing oligonucleotides suppress the expression of a gene whose expression or overexpression is associated with an ocular disease or disorder. In a certain embodiment, the gene silencing/editing oligonucleotides suppress the expression of the IL-1β, TNFα, COX-2, HIF-1α, VEGF-A, VEGF-B, PIGF, VEGFR1, VEGFR2, FGF-b, A-RAF, mTOR, MMM-2, MMP-9, and/or Integrin avb3 gene. In another embodiment, the gene silencing/editing oligonucleotides suppress the expression of mutant allele(s) associated with a dominant retinal disorder. In a further embodiment, the recombinant AAV expresses a transgene that encodes the wild-type gene. In a particular embodiment, the dominant retinal disorder is retinitis pigmentosa. In a further embodiment, the outer coating comprising hyaluronic acid is contacted with the one or more acid labile degradable polymer layers through electrostatic interactions. In an alternate embodiment, the outer coating comprising hyaluronic acid is contacted with the one or more acid labile degradable polymer layers through covalent bonds.

In a certain embodiment, the disclosure also provides for a pharmaceutical composition which comprises a hyaluronic acid functionalized chimeric viral/nonviral nanoparticle disclosed herein. In a further embodiment, the pharmaceutical composition is formulated for administration by intravitreal injection, parenterally, or by subretinal injection.

In a particular embodiment, the disclosure further provides for a method of treating a subject that has an ocular disease or disorder, comprising: administering to the subject an effective amount of a hyaluronic acid functionalized chimeric viral/nonviral nanoparticle disclosed herein. Examples of ocular diseases or disorders includes, but are not limited to, age-related macular degeneration, retinitis pigmentosa, Stargardt disease, Usher syndrome, rod-cone dystrophy, Bardet-Biedl syndrome, diabetic retinopathy, choroideremia, Oguchi disease, malattia leventinese, intraocular cancer, retinoblastoma, central retinal vein occlusion, branched retinal vein occlusion, blue-cone monochromacy, albinism, bacterial keratitis, chorioretinopathy, glaucoma, conjunctivitis, cytomegalovirus retinitis, drusen, Fuchs' dystrophy, fungal keratitis, viral keratitis, macular telangiectasia, optical neuritis, and scleritis. In a further embodiment, the ocular disease or disorder is a retinal disease or disorder selected from the group consisting of age-related macular degeneration, retinitis pigmentosa, Stargardt disease, Usher syndrome, rod-cone dystrophy, Bardet-Biedl syndrome, diabetic retinopathy, choroideremia, Oguchi disease, malattia leventinese, intraocular cancer, retinoblastoma, central retinal vein occlusion, branched retinal vein occlusion, and blue-cone monochromacy. In a particular embodiment, the ocular disease or disorder is age-related macular degeneration. In another embodiment, the hyaluronic acid functionalized chimeric viral/nonviral nanoparticle is administered in combination with an ophthalmological or eye treatment. In yet another embodiment, the ophthalmological or eye treatment is selected from bevacizumab, aflibercept, anecortave, pegaptanib, ranibizumab, verteporfin, interferon, ciprofloxacin, prednisolone acetate, ofloxacin, neomycin, polymyxin B, dexamethasone, trimethoprim sulfate, tobramycin, gentamicin, moxifloxacin, sulfacetamide, gatifloxacin, besifloxacin, loteprednol, azithromycin, natamycin, or any combination thereof.

In a certain embodiment, the disclosure further provides for a method of treating a subject that has a liver disease or disorder, comprising: administering to the subject an effective amount of a hyaluronic acid functionalized chimeric viral/nonviral nanoparticle disclosed herein. Examples of liver diseases or disorders includes, but are not limited to, hepatitis A, hepatitis B, hepatitis C, fatty liver disease, liver cancer, Wilson disease, hemochromatosis, Alagille syndrome, alcohol-related liver disease, alpha-1 antitrypsin deficiency, autoimmune hepatitis, biliary atresia, cirrhosis, Crigler-Najjar Syndrome, Galactosemia, Gilbert Syndrome, hepatic encephalopathy, hepatorenal syndrome, lysosomal acid lipase deficiency, non-alcoholic fatty liver disease, non-alcoholic steatohepatitis, primary biliary cholangitis, primary sclerosing cholangitis, Reye syndrome, Type I glycogen storage disease, hemophilia A and hemophilia B. In a further embodiment, the core comprises an AAV5, AAV8, AAVrh10 or AAV6. In yet a further embodiment, the AAV vectors further comprise liver specific protomers, such as two copies of alpha 1 microglobulin/bikunin enhancer coupled to the core promoter of human thyroxine-binding globulin (TBG). In yet a further embodiment, the AAV vectors further comprise a woodchuck hepatitis virus post-transcriptional regulatory element (WPRE). In yet another embodiment, the AAV vectors express from a heterologous transgene to treat a liver disease or disorder. Examples of such transgenes, include but are not limited, to transgenes that express a wild type gene for factor IX (FIX), FVIII gene, hAAt, OTC gene, LDL receptor gene, PBGD gene, Padua mutant factor IX gene, ARSB gene, and UGT1A1 gene. In another embodiment, the ChNP polymer layers comprise encapsulated gene silencing/editing oligonucleotides that are used to treat a liver disease or disorder. Such silencing/editing oligonucleotides include, but are not limited, to suppressing the expression of mutant alleles associated with a disorder (e.g., suppressing Z-AAT for liver disease associated with an AAT deficiency), LDL receptors, ApoB-100, proprotein convertase subtilisin/kexin type 9 (PCSK9), Fas-mediated apoptosis, and miRNAs associated with hepatic lipid metabolism, (e.g., miR-122).

### DESCRIPTION OF DRAWINGS

FIG. 1 presents an embodiment for the synthesis of viral/nonviral chimeric nanoparticles (ChNPs) of the disclosure. As shown, AAV is encapsulated with an acid-degradable polyketal (PK) shell via photopolymerization of acid-cleavable amino ketal monomers and cross-linkers in the presence of eosin, a photoinitiator. The PK shell is synthetically programmed to degrade in a mildly acidic environment (e.g., environment found in endosome/lysosome). Prior to the polymerization, siRNA can be premixed with the amino ketal monomers for concurrent encapsulation in the PK shell, thereby releasing siRNA and AAV to affect intracellular processes when taken up by lysosomes.

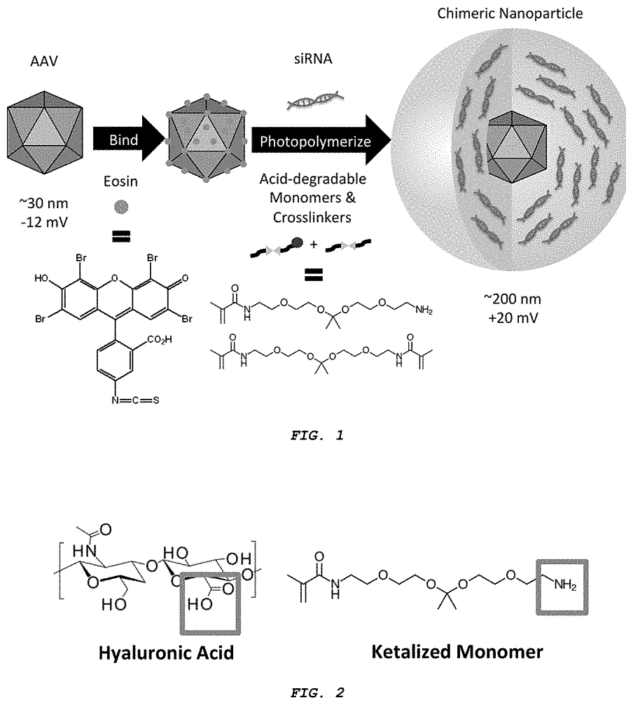

FIG. 2 shows an embodiment of the structure of hyaluronic acid (HA) and the ketal monomer that can be used with acid-degradable polyketal shell of the ChNPs. The boxes indicate the carboxylic acid group of HA molecule that is crosslinked via amide bond formation with the primary amine of the ketal monomer.

FIG. 3A-B presents size and zeta potential of ChNPs functionalized with or without HA. (A) Presents the sizes of ChNPS with and without HA as found by dynamic light scattering using a Malvern Zetasizer and deionized water as a solvent. (B) Presents the zeta potentials of ChNPS with and without HA as found by dynamic light scattering using a Malvern Zetasizer and deionized water as a solvent. ChNPs are positively charged while HA is negatively charged.

FIG. 4 presents the zeta potential of ChNPs functionalized with or without HA that have been further treated with or without an added base, sodium hydroxide. A change in zeta potential from the negative to the positive after treatment with the base indicates that HA is electrostatically bound to ChNPs. If the zeta potential remains negative after treatment with the base then it indicates that HA is covalently bound to ChNPs.

FIG. 5 provides the results of toxicity studies with AAV, AAV-HA, ChNP, ChNP-HA, ChNP/HA on retinal ARPE-19 cells. ChNP-HA, refers to HA covalently bound to ChNP; ChNP/HA, refers to HA electrostatically bound to ChNP; and AAV-HA, refers to HA covalently bound to ChNP. As shown, ChNP-HA and ChNP/HA were far less toxic to retinal cells than ChNP.

FIG. 6 presents the transduction efficiency of AAV, AAV-HA, ChNP, ChNP-HA, ChNP/HA on retinal ARPE-19 cells. ChNP-HA, refers to HA covalently bound to ChNP; ChNP/HA, refers to HA electrostatically bound to ChNP; and AAV-HA, refers to HA covalently bound to ChNP. As shown, ChNP-HA was superior to ChNP and ChNP/HA in transfecting retinal cells.

FIG. 7 presents fluorescent and brightfield images that were obtained and quantified using GUAVA flow cytometry of ChNPs functionalized with and without HA, and AAV. ChNPs and AAV were delivered to each well and incubated overnight at 37° C., in concentrations of 2e10 GCs/mL. The next day media was replaced with fresh media. The images were taken on days 3, 4, and 5 (post-treatment).

FIG. 8 presents fluorescent and brightfield images that were obtained and quantified using GUAVA flow cytometry of ChNPs functionalized with and without HA and AAV. ChNPs and AAV were delivered to each well and incubated overnight at 37° C., in concentrations of 1e10 GCs/mL. The next day media was replaced with fresh media. The images were taken on days 3, 4, and 5 (post-treatment).

FIG. 9 presents fluorescent and brightfield images that were obtained and quantified using GUAVA flow cytometry of ChNPs functionalized with and without HA and AAV. HA and AAV were delivered to each well and incubated overnight at 37° C., in concentrations of 5e9 GCs/mL. The next day media was replaced with fresh media. The images were taken on days 3, 4, and 5 (post-treatment).

FIG. 10 presents sectioned retina images from mice that were intravitreally injected with GFP-ChNP (top panel) or GFP-ChNP-HA (lower panel). The images were from 7 days post treatment. An anti-GFP antibody labeled with Alexa Fluor 633 was used to visualize the location of the ChNPs (pink in the images). As shown, ChNP-HA were localized in the inner retinal cells (lower panel), while there was no such localization by ChNP (upper panel).

FIG. 11 presents the results of stability experiments looking at changes in the sizes and zeta potentials of ChNPs with and without HA after freezing, lyophilization, and reconstitution.

FIG. 12 presents the results of stability experiments looking at changes in transduction efficiencies of ChNPs functionalized with and without HA after freezing, lyophilization, and reconstitution.

FIG. 13 presents fluorescent and brightfield images that were obtained and quantified using GUAVA flow cytometry of samples of post-freeze dried ChNPs functionalized with and without HA and AAV. HA and AAV were delivered to each well and incubated overnight at 37° C., in concentrations of 2e10 GCs/mL. The next day media was replaced with fresh media. The images were taken on days 3, 4, and 5 (post-treatment).

FIG. 14 presents fluorescent and brightfield images that were obtained and quantified using GUAVA flow cytometry of samples of post-freeze dried ChNPs with and without HA and AAV. HA and AAV were delivered to each well and incubated overnight at 37° C., in concentrations of 1e10 GCs/mL. The next day media was replaced with fresh media. The images were taken on days 3, 4, and 5 (post-treatment).

FIG. 15 presents fluorescent and brightfield images that were obtained and quantified using GUAVA flow cytometry of samples of post-freeze dried with and without HA and AAV. HA and AAV were delivered to each well and incubated overnight at 37° C., in concentrations of 5e9 GCs/mL. The next day media was replaced with fresh media. The images were taken on days 3, 4, and 5 (post-treatment).

### DETAILED DESCRIPTION

As used herein and in the appended claims, the singular forms “a”, “an”, and “the” include plural referents unless the context clearly dictates otherwise. Thus, for example, reference to “an AAV” includes a plurality of such AAVs and reference to “the ketal monomer” includes reference to one or more ketal monomers and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

There are a variety of ocular diseases that have limited to no effective medical treatments. Many ocular diseases result from excessive neovascularization (NV), an abnormal proliferation and growth of blood vessels within the eye. The development of ocular NV itself has adverse consequences for vision but also is an early pathological step in many serious eye diseases. Despite introduction of new therapeutic agents against NV, NV remains the most common cause of permanent blindness in United States and Europe. Several major eye diseases give rise to abnormal neovascularization, which leads to further damage to the eyes causing loss of vision. Unfortunately, few treatment options exist for patients with these ocular NV diseases. The most common approved therapy is a photodynamic treatment, Visudyne, that uses light to activate a photosensitizer in the vicinity of the neovascularization to destroy unwanted blood vessels. It is not effective in many patients and cannot prevent recurrence even when it is effective. A recently approved agent, Macugen, provides some benefit but also is ineffective in most patients. Intraocular administration of Macugen can lead to irritation and risk of infection, both of which are adverse since they exacerbate the neovascularization pathology. As a consequence, more effective treatments are needed to prevent ocular disease progression and/or to treat the underlying ocular disease itself.

The National Eye Institute of NIH has estimated, 400,000 Americans have had some form of ocular herpes, and there are nearly 50,000 new cases diagnosed each year in the United States, with the more serious stromal keratitis accounting for about 25%. From a larger study, it was found that the recurrence rate of ocular herpes is 10 percent in one year, 23 percent in two years, and 63 percent within 20 years. Although application of available anti-viral drugs could control the HSV infection to certain extent, there is no effective medication available that could treat the HSV-caused stromal keratitis which would protect the patients from blindness.

The ocular neovascularization diseases can be divided into diseases affecting the anterior, or front, of the eye and those affecting the posterior, or retinal, part of the eye. Development of NV at these different regions may have different origins, but the biochemical and physiological nature of the NV process appears to be virtually identical, regardless of eye region. Consequently, an effective means to intervene in the biochemical nature of ocular NV offers the prospect for providing an effective treatment for any ocular disease that involves ocular NV as the major pathology or as the underlying pathology, regardless of whether the disease afflicts the anterior or posterior of the eye. Nonetheless, the anterior and posterior ocular tissues differ considerably and these differences can have a dramatic influence on the most effective means to administer therapeutic treatments so that the tissue and cells are reached by the therapeutic agent.

Like other tissues, ocular tissues are in a continuous state of maintenance which often entail neovascularization. At late stage of most ocular diseases, ocular neovascularization becomes a major symptom of the diseases. Most treatments are directed to correcting this abnormal physiological change. Moreover, ocular neovascularization appears to be virtually identical regardless of the region of the eye and disease and irrespective of the originating cause of the pathology. This commonality of the pathological neovascularization process provides an ideal intervention target for developing therapies against diseases of the eye.

The present disclosure provides for hyaluronic acid coated viral/nonviral nanoparticles that have multimodal effectiveness against various ocular diseases and disorders by comprising a viral core that can be used for gene therapy surrounded by an outer polymeric shell that comprises therapeutics (e.g., siRNA) which has been further coated with hyaluronic acid so as to promote uptake of the particles by ocular cells (e.g., retinal cells). Gene therapy is usually performed with viral or nonviral vectors to deliver desired nucleic acids. By combining viral and nonviral platforms into a hybrid therapy, one can take advantage of immune masking, leading to greater biocompatibility, while maintaining efficient viral gene delivery. With a hybrid delivery vehicle, the carrier can be modified to contain both a virus and other nucleic acids or therapeutic agents. This multi-modal therapy can attack pathological-associated biological genes/pathways at multiple points, leading to a synergistic therapeutic effect.

Adeno-associated virus (AAV) has been used increasingly as a promising vector for gene therapy. AAV is a small, nonenveloped virus that can transduce both dividing and quiescent cells, making it useful for many applications in gene therapy. The small size allows for surface modifications or encapsulation and is ideal for drug delivery. A host's immune response to AAV is mostly limited to neutralizing antibodies, which leads clearance, but no side effects. AAV's genome stably integrates into a specific site on chromosome 19, ridding it of oncogenesis concerns.

AAV is capable of transducing multiple cell types within the retina. AAV serotype 2 (AAV2), the most well-studied type of AAV, is commonly administered in one of two routes: intravitreal or subretinal. Using the intravitreal route, AAV is injected in the vitreous humor of the eye. Using the subretinal route, AAV is injected underneath the retina, taking advantage of the potential space between the photoreceptors and RPE layer, in a short surgical procedure. Although this is more invasive than the intravitreal route, the fluid is absorbed by the RPE and the retina flattens in less than 14 hours without complications. Intravitreal AAV targets retinal ganglion cells and a few Muller glial cells. Subretinal AAV efficiently targets photoreceptors and RPE cells. Following intraocular administration, AAV2 gives rise to a minimal systemic immune response, and neutralizing antibodies against the AAV capsid are only detected in serum following treatment with a high dose and are not sufficient to attenuate transgene expression. There is also no evidence of an antibody-mediated response against transgene products following long-term AAV-mediated expression, and animals previously injected with AAV in one eye show evidence of reporter gene expression in the fellow eye following repeat vector administration (RA, unpublished data). This excellent track record of safety allows long-term expression in dogs and nonhuman primates for up to 3 years, and makes rAAV the vector of choice for stable, safe and efficient gene transfer to the eye in clinical applications.

The reason that different routes of administration lead to different cell types being transfected (e.g., different tropism) is that the inner limiting membrane (ILM) and the various retinal layers act as physical barriers for the delivery of drugs and vectors to the deeper retinal layers. Thus overall, subretinal AAV is 5-10 times more efficient than delivery using the intravitreal route.

Initial studies with AAV in the retina have utilized AAV serotype 2. Researchers are now beginning to develop new variants of AAV, based on naturally-occurring AAV serotypes and engineered AAV variants.

Several naturally-occurring serotypes of AAV have been isolated that can transduce retinal cells. Following intravitreal injection, only AAV serotypes 2 and 8 were capable of transducing retinal ganglion cells. Occasional Muller cells were transduced by AAV serotypes 2, 8, and 9. Following subretinal injection, serotypes 2, 5, 7, and 8 efficiently transduced photoreceptors, and serotypes 1, 2, 5, 7, 8, and 9 efficiently transduce RPE cells. Newly isolated serotypes deriving from humans (AAVhu29R, AAV7, AAV8 and AAV9) and from rhesus macaques (AAVrh.43 and AAV64R1) have been used to package AAV2-based genomes and the novel pseudotypes compared to AAV2/5 for their ability to transduce photoreceptors. One example of an engineered variant has recently been described that efficiently transduces Muller glia following intravitreal injection, and has been used to rescue an animal model of aggressive, autosomal-dominant retinitis pigmentosa.

Importantly, the retina is immune-privileged, and thus does not experience a significant inflammation or immune-response when AAV is injected. Immune response to gene therapy vectors is what has caused previous attempts at gene therapy to fail, and is considered a key advantage of gene therapy in the eye. Re-administration has been successful in large animals, indicating that no long-lasting immune response is mounted. Recent data indicates that the subretinal route may be subject to a greater degree of immune privilege compared to the intravitreal route.

Expression in various retinal cell types can be determined by the promoter sequence. In order to restrict expression to a specific cell type, a tissue-specific or cell-type specific promoter can be used. For example, in rats the murine rhodopsin gene drive the expression in AAV2, GFP reporter product was found only in rat photoreceptors, not in any other retinal cell type or in the adjacent RPE after subretinal injection. On the other hand, if ubiquitously expressed immediate-early cytomegalovirus (CMV) enhancer-promoter is expressed in a wide variety of transfected cell types. Other ubiquitous promoters such as the CBA promoter, a fusion of the chicken-actin promoter and CMV immediate-early enhancer, allows stable GFP reporter expression in both RPE and photoreceptor cells after subretinal injections.

One important factor in gene delivery is developing altered cell tropisms to narrow or broaden rAAV-mediated gene delivery and to increase its efficiency in tissues. Specific properties like capsid conformation, cell targeting strategies can determine which cell types are affected and also the efficiency of the gene transfer process. Different kinds of modification can be undertaken. For example, modification by chemical, immunological or genetic changes that enables the AAV2 capsid to interact with specific cell surface molecules.

Modification of AAV can be achieved with both polymeric materials as well as natural ones. For example, eosin can be bound to the surface of AAV, which along with ascorbic acid, is used to form polymers via photo-polymerization of monomers (e.g., ketal monomers). Other agents, like therapeutic agents can be added to photo-polymerization reaction as well. For example, siRNA may be incorporated into the polymers. Therefore, AAV particles comprising polymer shell(s) can be multimodal to combat a disease or disorder at multiple cellular levels, e.g., the AVV core can provide gene therapy while the polymeric shell can be used to deliver one or more therapeutic agents. Moreover, the ketal-based polymers are susceptible to hydrolysis in the presence of weak acid environment, like in a lysosome. Accordingly, with the hydrolysis of the polymers, the cargo (e.g., therapeutic agent and AAV core) will be released inside the cell.

The chimeric viral/nonviral nanoparticles (ChNPs) described herein have been further functionalized on the outer surface with hyaluronic acid. Hyaluronic acid (HA) may be affixed to the ChNPs using covalent or noncovalent interactions (e.g., electrostatic interactions). HA is an anionic biodegradable, non-immunogenic biopolymer which is ubiquitously present in mammalian organisms. It is a nonsulfated glycosaminoglycan, composed of alternating disaccharide units of N-acetyl-D-glucosamine and D-glucuronic acid, linked by alternating β-1,4 glycosidic and β-1,3 glucuronidic bonds. HA is present in the extracellular matrix, and plays an important role in cell proliferation, differentiation, motility, adhesion and gene expression. HA can be efficiently taken up by cells through CD44 receptor-mediated endocytosis. HA has been used as drug carrier, and ligand on various nanoparticles. HA is a major constituent of vitreous humor, is found throughout the retina and many retinal cell types have been shown to express CD-44 receptors on their surface.

Achieving efficient, long-term expression of a transgene following intraocular delivery offers the means to target many life-long retinal disorders. Most forms of inherited retinal diseases are caused by mutations in genes expressed in rod and cone photoreceptors and in the retinal pigment epithelium (RPE). AAV is the only viral vector to efficiently transduce both RPE and photoreceptors. This is probably due to a combination of factors. Photoreceptors express the specific receptors required for the binding of some types of AAV. Also the inter-photoreceptor matrix and the outer limiting membrane, protein components of the neurosensory retina, represent physical barriers that prevent larger virus particles, such as HIV (around 120 nm in size) gaining access to photoreceptor cells, whereas the smaller size of mature AAV virions (around 20 nm) may allow outer retinal neurons to be transduced. Intravitreal delivery of AAV leads to efficient ganglion cell transduction, offering the potential to deliver therapeutic genes to cells of the inner retina. These features mean that AAV is regarded as the vector of choice for gene therapies aimed at inherited retinal disorders and for acquired disorders, such as AMD.

In a particular embodiment, the disclosure provides for a HA functionalized ChNPs which comprises a recombinant AAV core (of a serotype, as described above) which expresses a gene therapy product from a heterologous transgene that can be used to treat an ocular disease or disorder, such as by increasing the expression of genes that are poorly expressed or not expressed due to an inherited retinal disorder or suppressed due to later developing disorder, like AMD. Examples of gene therapy products that can be encoded by the heterologous transgenes, includes transgenes that comprise a wild type gene RPE65 gene (RPE65 is an isomerohydrolase expressed in retinal pigment epithelium), a REP (Rab escort protein-1) gene, an RS1 (retinoschisin) gene, a ciliary neurotrophic factor (CNTF) gene and/or a PEDF (Pigment epithelium-derived factor) gene.

Additionally, in dominant retinal disorders, such as retinitis pigmentosa caused by mutations in Rhodopsin, gene replacement is not sufficient to overcome the expression of the mutant allele. In this case, therapies that ablate mutant transcripts, and then replace them with wild-type genes, are required. Knockdown of mRNA can be achieved using ribozymes, or gene silencing/editing oligonucleotides. Designing unique interfering RNA molecules specific to each mutant allele is not feasible as there are over 100 dominant mutant alleles of Rhodopsin alone. The ideal RNAi-based strategy may be to target a 5′ untranslated region of the gene of interest, leading to the cleavage of all the transcripts for the target gene (including wild-type as well as mutant transcripts), in combination with the delivery of a wild-type gene. Alternatively, it is possible to target part of the coding sequence independent of the mutation, in combination with delivery of a wild-type sequence engineered to be resistant to degradation using the degeneracy of the genetic code. Extensive studies have demonstrated the feasibility of vector-mediated RNA interference in the central nervous system, using AAV-mediated expression of a small hairpin RNA (shRNA) that is processed intracellularly to an active form. AAV. shRNA delivery mediates improvements in motor neuron function and in neuronal morphology for at least 21 weeks in murine models of degeneration in the central nervous system. Following studies showing that AAV. shRNA delivery reduces Rhodopsin expression in vitro, a recent report shows that in vivo expression of a human Rhodopsin transgene can be reduced by up to 90% and that a nonsilenced Rhodopsin gene can be expressed to achieve a degree of rescue. Eyes treated with the suppression-replacement construct showed some preservation of photoreceptors, indicating this approach may be useful in treating dominantly inherited retinal degenerations. The HA functionalized ChNPs of the disclosure are ideally suited to treating such dominant retinal disorders, as the AAV portion of the nanoparticle can express the wild type gene product from a from a transgene (e.g., a Rhodopsin transgene), while simultaneously providing ribozymes, or gene silencing/editing oligonucleotides encapsulated in the polymer layers that can be used to suppress mutant allele expression. Suppression-replacement strategies for treating dominant retinal disorders can be realized by use the HA functionalized ChNPs of the disclosure.

In a another embodiment, the disclosure provides for a HA functionalized ChNPs which comprises a recombinant AAV core (of a serotype, as described above) which expresses a gene therapy product from a heterologous transgene that can be used to treat a liver disease or disorder, such as by increasing the expression of genes that are poorly expressed or not expressed due to damage caused to the liver by viruses, alcohol consumption, obesity, diabetes, and/or and inheritable condition (e.g., lack of factor IX (FIX) in hemophilia patients). Liver-directed gene therapy using AVV vectors to treat diseases like hemophilia, Crigler Najjar, Wilson disease, OTC deficiency, GSDla, PKU, Citrullinemia type 1, and methylmalonic acidemia, have been developed and are being tested in clinical trials (e.g., see Kattenhorn et al., Human Gene Therapy 27(12):947-961 (2016)). The most common AAV serotype for these gene therapies, include AAV5, AAV8, AAVrh10, and AAV6. Numerous studies in classic mouse and dog models of hemophilia A and B have demonstrated clear and robust long-term benefit from administration of AAV vectors encoding the relevant clotting factors, with the vector trafficking to the liver for gene expression. Further, these AAV vectors may further comprise liver specific promoters like two copies of alpha 1 microglobulin/bikunin enhancer coupled to the core promoter of human thyroxine-binding globulin (TBG). Expression can be further stabilized by the inclusion of a woodchuck hepatitis virus post-transcriptional regulatory element (WPRE). Examples of gene therapy products that can be encoded by the heterologous transgenes for liver diseases or disorders, includes transgenes that comprise a wild type gene for factor IX (FIX), FVIII gene, hAAt, OTC gene, LDL receptor gene, PBGD gene, Padua mutant factor IX gene, ARSB gene, and UGT1A1 gene. Examples of liver diseases or disorders includes, but are not limited to, hepatitis A, hepatitis B, hepatitis C, fatty liver disease, liver cancer, Wilson disease, hemochromatosis, Alagille syndrome, alcohol-related liver disease, alpha-1 antitrypsin deficiency, autoimmune hepatitis, biliary atresia, cirrhosis, Crigler-Najjar Syndrome, Galactosemia, Gilbert Syndrome, hepatic encephalopathy, hepatorenal syndrome, lysosomal acid lipase deficiency, non-alcoholic fatty liver disease, non-alcoholic steatohepatitis, primary biliary cholangitis, primary sclerosing cholangitis, Reye syndrome, Type I glycogen storage disease, hemophilia A and hemophilia B. HA has also been used to target therapeutics to the liver, as liver sinusoidal endothelial cells comprise hyaluronic acid receptor for endocytosis (HARE) which promotes endocytosis of HA, as well as, heparin, dermatan sulfate, and acetylated low-density lipoprotein. HARE mediates systemic clearance of hyaluronan and chondroitin sulfates from the vascular and lymphatic circulations. The internalized glycosaminoglycans are degraded in lysosomes, thus completing their normal turnover process. As such, the HA functionalized ChNPs are ideally suited for using liver endocytic processes, as the ChNP polymer layers are designed to degrade in acid environments, such as those found in lysosomes. Further, these polymer layers may contain therapeutics and drug products that have been used to treat liver diseases or disorders, such as chemotherapeutics for liver cancer, corticosteroids, ursodiol, immunomodulators, and antiviral medications. Alternatively, or in addition, the polymer layers may comprise encapsulated gene silencing/editing oligonucleotides that are used to treat a liver disease or disorder, such sequences can be directed to suppressing mutant alleles associated with a disorder (e.g., suppressing Z-AAT for liver disease associated with an AAT deficiency), LDL receptors, ApoB-100, proprotein convertase subtilisin/kexin type 9 (PCSK9), Fas-mediated apoptosis, and miRNAs associated with hepatic lipid metabolism, (e.g., miR-122).

A hyaluronic acid functionalized ChNP of the disclosure can be administered to any host, including a human or non-human animal, in an amount effective to treat a disease or disorder disclosed herein. Thus, the methods and compositions of the disclosure are useful as multimodal therapies for treating diseases and disorders by expressing a transgene that can use for gene therapy while delivering an additional therapeutic to treat the same disease or disorder, or to inhibit biological activities that are associated with the disease or disorder such as inflammation, swelling, immune response, etc. The additional therapeutics can be encapsulated by the one or more acid labile polymer layers of the hyaluronic acid functionalized ChNP and can include nucleic acids (e.g., siRNAs, shRNAs, miRNAs, DNA, cDNA), CRISPR-Cas or CRISPRi systems, therapeutic proteins, small molecule therapeutics (e.g., ophthalmologicals, eye treatments, liver treatments), etc. In a particular embodiment, the one or more acid labile polymer layers of the hyaluronic acid functionalized ChNPs comprise siRNAs, miRNAs, or shRNAS. Targets for the gene silencing/editing oligonucleotides can include genes and their products which are associated with ocular diseases and disorders, such as growth factors, metalloproteins, and viruses (e.g., see Table 1).

TABLE 1
Ocular target genes for RNAi

| Organism | Gene | Accession No. |
|---|---|---|
| HSV-1 | UL5 | DQ889502 |
| HSV-2 | UL5 | NC_001798 |
| HSV-1 | UL29 | DQ889502 |
| HSV-2 | UL29 | NC_001798 |
| Human | IL-1β | NM_000576 |
| Human | TNFα | NM_000594 |
| Human | COX-2 | AY462100 |
| Human | HIF-1α | NM_001530 |
| Human | VEGF-A | NM_001025366 |
| Human | VEGF-B | NM_003377 |
| Human | PIGF | NM_002643 |
| Human | VEGFR1 | BC039007 |
| Human | VEGFR2 | NM_010612 |
| Human | FGF-b | NM_002006 |
| Human | A-RAF | NM_001654 |
| Human | mTOR | L34075 |
| Human | MMP-2 | NM_004530 |
| Human | MMP-9 | NM_004994 |
| Human | Integrin avb3 | NM_002210 |

The siRNAS can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Göttingen, Germany, and can be found by accessing the website of the Max Planck Institute and searching with the keyword “siRNA.” Thus, the sense strand of the present siRNA comprises a nucleotide sequence identical to any contiguous stretch of about 19 to about 25 nucleotides in the target mRNA. Generally, a target sequence of the target mRNA can be selected from a given cDNA sequence corresponding to the target mRNA (e.g., the mRNA sequences for the genes listed in Table 1).

In a particular embodiment, the disclosure provides for one or more acid labile degradable polymer layers that surround the core of the nanoparticle. Ideally, the acid labile degradable polymer layers will degrade in mildly acidic environments found in endosomes (pH 5.0-6.8) or lysosomes (pH 4.5-5.5). Examples of such polymers, include those based upon polyketals, poly(amido amine)s, diacetals, and poly(2-(diethylamino)ethyl methacrylate) (PDEAEMA). In a particular embodiment, the disclosure provides that the one or more acid labile degradable polymer layers are polyketal-based polymer layers. As used herein, a “polyketal” refers to a homo- or co-polymer that includes two or more (i.e., a plurality) of ketal repeat units. As used herein, a “ketal” repeat unit is a unit including a ketal-containing group that is repeated in the polymer at least once. A ketal group is a group that includes an —O—C(M) (N)—O— functionality with the proviso that neither M nor N is hydrogen (e.g., an acetal-containing group) or oxygen (e.g., an orthoester-containing group). Methods for preparing such polyketal polymers can be found herein, and in U.S. Pat. No. 7,741,375, Yang et al., Bioconjugate Chem. 19(6):1164-1169 (2008), Heffernan et al., Bioconjugate Chem. 16(6):1340-1342 (2005), Louage et al., Biomacromolecules 16(1):336-350 (2015), the disclosures of which are incorporated herein by reference.

Any of a variety of art-known methods can be used to administer a HA functionalized ChNP disclosed herein either alone or in combination with one or more additional chemotherapeutic agents. For example, administration can be parenterally, by injection or by gradual infusion over time. The HA functionalized ChNPs alone or with additional therapeutic agents can be administered intravenously, intraperitoneally, intramuscularly, subcutaneously, intracavity, by inhalation, or transdermally.

Preparations for parenteral administration of a composition comprising a HA functionalized ChNP of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

Generally, the optimal dosage of the HA functionalized ChNPs will depend upon the type and stage of the disease or disorder and factors such as the weight, sex, and condition of the subject. Nonetheless, suitable dosages can readily be determined by one skilled in the art. Typically, dosages used in vitro may provide useful guidance in the amounts useful for in situ administration of the pharmaceutical composition, and animal models may be used to determine effective dosages for treatment of specific infections. Various considerations are described, e.g., in Langer, Science, 249: 1527, (1990); Gilman et al. (eds.) (1990), each of which is herein incorporated by reference. Typically, a suitable dosage for HA functionalized ChNPs is 1 to 1000 mg/kg body weight, e.g., 10 to 500 mg/kg body weight. In a particular embodiment, a HA functionalized ChNP disclosed herein is administered at dosage of 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 100 mg/kg, or a range that includes or is between any two of the foregoing dosages, including fractional dosages thereof.

A pharmaceutical composition comprising a HA functionalized ChNP of the disclosure can be in a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The disclosure further provides for a pharmaceutical composition comprising a hyaluronic acid functionalized ChNP that is administered by an intravitreal injection, parenterally, or by a subretinal injection. In particular embodiment, the disclosure provides a pharmaceutical composition that comprises a hyaluronic acid functionalized ChNP disclosed herein that is used to treat a disease or disorder, such as an ocular disease or disorder, or a liver disease or disorder.

Intravitreal (IVT) injection is a widely-used technique to deliver therapeutic agents, like vascular endothelial growth factor inhibitors, antibiotics and glucocorticoids. IVT injections are one of the most commonly performed ocular surgery procedure in the developed world, second only to cataract surgery. The procedure is generally performed under local anesthesia with e.g., lidocaine 2%. During the procedure, the eyelids and eyelashes are treated with disinfectant such as a povidone-iodine solution. Subsequently, a 30 Gauge needle is inserted through the sclera at the pars plana region, 3.5-4 mm posterior to the limbus between vertical and horizontal muscles. The therapeutic agent is directly injected into the vitreous cavity with limited reflux. IVT injections bypass the blood retinal barrier so as to provide clinically effective doses of therapeutic agents to the target tissue. Therapeutic intraocular concentrations of the hyaluronic acid functionalized ChNPs can be achieved immediately and effectively without the danger of systemic absorption and toxicity.

Unlike IVT, subretinal (SR) injections constitute “proper” ophthalmic surgery performed by vitreo-retinal surgeons. SR injections are routinely used in severe cases of submacular hemorrhage or other complex vitreoretinal disease involving the subretinal space. In clinical research, subretinal surgery has been performed in macular translocation surgeries, electronic, or stem-cell implants and gene therapy trials, with the aim to prevent or reverse blindness. The SR injection can be performed under retro-/parabular anesthesia or under general anesthesia in an operating theater. After disinfection, a three-port pars plana vitrectomy is performed, mostly using standard 23 or 25G trocar systems. After successful detachment of the posterior hyaloid membrane and removal of the vitreous, e.g., a double-barreled 23G needle with 41G tip is inserted through the trocar. The tip is guided to the subretinal area and a small infusion of balanced salt solution (BSS) is performed into the potential subretinal space to form a bleb. Once the subretinal space has formed and location of the bleb is within the targeted region, the same retinotomy (injection channel through neuroretina) is used to guide a second instrument with the same tip built into the subretinal space for the injection of the therapeutic agent using a controlled flow rate.

Other considerations for ocular delivery of the HA functionalized ChNPs of the disclosure, including anatomical considerations, immune responses and vector re-administration, retinal adhesiveness, etc. can be considered as is detailed in Ochakovski et al., Front. Neurosci, 11:174 (2017), which is incorporated herein by reference.

The disclosure further provides for a pharmaceutical composition comprising a hyaluronic acid functionalized ChNP disclosed herein that can be administered by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The disclosure provides methods for inhibiting an ocular disease or disorder, by contacting or administering a therapeutically effective amount of a hyaluronic acid functionalized ChNP disclosed herein, either alone or in combination with other therapeutic agents to a subject who has, or is at risk of having, such an ocular disease or disorder. Examples of ocular diseases or disorder include, but are not limited to, age-related macular degeneration, retinitis pigmentosa, Stargardt disease, Usher syndrome, rod-cone dystrophy, Bardet-Biedl syndrome, diabetic retinopathy, choroideremia, Oguchi disease, malattia leventinese, intraocular cancer, retinoblastoma, central retinal vein occlusion, branched retinal vein occlusion, blue-cone monochromacy, albinism, bacterial keratitis, chorioretinopathy, glaucoma, conjunctivitis, cytomegalovirus retinitis, drusen, Fuchs' dystrophy, fungal keratitis, viral keratitis, macular telangiectasia, optical neuritis, and scleritis. In a particular embodiment, the hyaluronic acid functionalized ChNP disclosed herein, either alone or in combination with other therapeutic agents to a subject who has or is at risk of having a retinal disease or disorder. Examples of retinal diseases, disorders and conditions include, but are not limited to, age-related macular degeneration, retinitis pigmentosa, Stargardt disease, Usher syndrome, rod-cone dystrophy, Bardet-Biedl syndrome, diabetic retinopathy, choroideremia, Oguchi disease, malattia leventinese, intraocular cancer, retinoblastoma, central retinal vein occlusion, branched retinal vein occlusion, and blue-cone monochromacy. The disclosure further provides for use of a HA functionalized ChNPs of disclosure in combination with other agents, such as ophthalmologicals and eye treatments (e.g., antibiotics for eye infections). Examples of ophthalmologicals and eye treatments include, but are not limited to, bevacizumab, aflibercept, anecortave, pegaptanib, ranibizumab, verteporfin, interferon, ciprofloxacin, prednisolone acetate, ofloxacin, Maxitrol®, Polytrim®, Tobradex®, tobramycin, gentamicin, moxifloxacin, sulfacetamide, gatifloxacin, besifloxacin, Zylet®, Blephamide®, azithromycin, Tobradex ST®, Natacyn®, Pred-G®, and Bleph-10®. The disclosure further provides for use of a HA functionalized ChNPs disclosed herein in combination with an AMD treatment. Examples of AMD treatments include, but are not limited to, bevacizumab, aflibercept, anecortave, pegaptanib, ranibizumab, and verteporfin.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more HA functionalized ChNPs described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

Materials.

Eosin-5-isothiocyanate, Alexa Fluor 488 carboxylic acid succinimidyl ester, and Quant-iT PicoGreen nucleic acid assay kit are purchased from Invitrogen (Carlsbad, Calif.). N-Hydroxysuccinimide (NHS) and N,N-diisopropylethylamine (DIPEA) are purchased from Acros Organics (Thermo Fisher Scientific, Pittsburgh, Pa.), and 1-ethyl-3-(3-dimethylaminopropyl) carbodiimide (EDC) is purchased from Advanced ChemTech (Louisville, Ky.). (NHS)-functionalized polyethylene glycol (NHS-PEG, 5 kDA) are purchased from Creative PEG Works Inc. (Winston Salem, N.C., U.S.A.). PD10 size-exclusion column (MWCO 5 kDa) is purchased from GE Healthcare (Pittsburgh, Pa.) and Amicon Ultra Centrifugal filters (MWCO 100 kDa) are purchased from Millipore (Billerica, Mass.). QuickTiter AAV quantitation kit is purchased from Cell Biolabs (San Diego, Calif.). 3-(4,5-dimethyl-2-thiazolyl)-2,5-diphenyltetrazolium bromide (MTT) is purchased from Sigma Aldrich (St. Louis, Mo.) and nucleus-staining dye DRAQS is purchased from BioStatus (Leicestershire, UK). Anti-AAV polyclonal antibodies are purchased from IMGENEX (San Diego, Calif.). Acid-degradable amino ketal methacrylamide monomer and acid-degradable ketal bismethacrylamide cross-linker are synthesized as reported below, with slight modifications. Non-degradable cationic monomer and cross-linker, which contain an additional ethoxy group instead of ketal linkage, were also synthesized as reported below. Sialic acid is purchased from Nacalai USA (San Diego, Calif.).

Cell Culture.

HeLa cells were cultured in DMEM 10% FBS and 1% P/S. ARPE-19 cells were cultured in F12 DMEM 10% FBS, and 1% P/S. All cells were cultured at 37° C. with 5% CO₂ and 100% humidity.

Preparation of Chimeric Nanoparticles (ChNPs).

ChNPs were synthesized as described in Hong et al. (ACS Nano 10:8705-8714 (2016)). Briefly, heterologous gene-encoding AAV vectors (1.0×10¹¹ genome copy [GC]) in 5 mL of 10 mM sodium bicarbonate buffer (pH 8.0) are reacted with 2 mg of eosin-5-isothiocyanates in 10 μL of dimethyl sulfoxide (DMSO) with mild agitation. After 3 h incubation at RT, the residual eosin-5-isothiocyanates are removed using a PD mini size-exclusion column. The eosin-conjugated AAV vectors (6.0×10¹⁰ GC) are suspended in 1 mL of 10 mM HEPES buffer (pH 7.4) containing 10 mg of ascorbic acids. Ten mg of amino ketal methacrylamide monomers and 3.0 μg of siRNA are premixed in 50 μL of 10 mM HEPES buffer for 30 min at RT. The resulting monomers/siRNA solution is then added to eosin-conjugated AAV solution, followed by photopolymerization with mild stirring under a halogen lamp at 700 klux. After 10 min, 10 mg of amino ketal methacrylamide monomers and 4 mg of ketal bismethacrylamide cross-linkers are added and further polymerized for another 5 min. Ascorbic acids, unreacted monomers, and cross-linkers are removed by centrifugal filtration (100 kDa MWCO) of the resulting solution at 3000 rpm for 30 min at 4° C.

Functionalization of ChNPs with Hyaluronic acid (HA).

HA has been shown to increase the efficacy of nanoparticles to cross the retina in previous studies. Therefore, there is potentially to bind HA to the surface of ChNPs or viruses themselves in order to deliver genes to retinal cells. Through binding HA to ChNPs, these could be used to treat retinal diseases. Further, other studies have shown that HA can increase the efficacy of nanoparticles for treating cancer (e.g., see Vangara et al., Anticancer Research 33(6):2425-2434 (2013)).

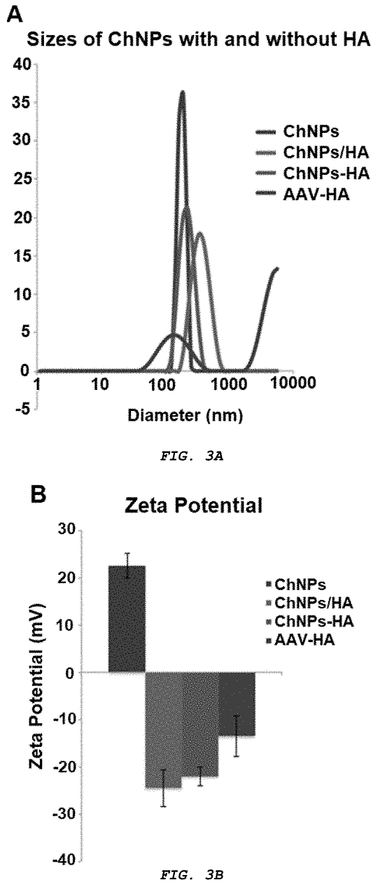

HA is a negatively charged polymer that could associate with positively charged ChNPs through electrostatic interactions. To make ChNPs/HA (electrostatically bound) 1 mg/mL of HA (85 kDa) was added to 6e10 GCs/mL ChNPs, and incubated for 30 min at room temperature. EDC chemistry is used to bind the carboxylic acid group on HA to the primary amine of the ketal monomer used to make up the ChNPs (e.g., see FIG. 2). To create ChNPs-HA and AAV-HA, first the HA (85 kDa) was activated. This was done through suspending 1 mg of HA in 1 mL of 0.1 mM sodium borate (pH=8), then adding 1 molar equivalent of EDC and 2 molar equivalents of NHS. This was allowed to initialize at room temperature for five minutes. Then 6e10 GCs of ChNPs (with AAV-GFP) or AAV-GFP were added into the EDC mixture to create ChNPs-HA or AAV-HA respectively. These were mixed overnight at room temperature. Afterwards the mixture was purified through 100 kDa centrifugal filtration at 3000 rpm, 30 min at 4° C., resuspended in 1 mL nuclease free H₂O and repeated, before final suspension in 1 mL H₂O.

Statistical Analysis.

All triplicate experimental data collected from independently repeated measurements are represented as mean±standard deviation. Statistical analysis is performed with Student's t Test and statistical significance is at p-values lower than 0.05.

Characterization of ChNPs/HA (Electrostatic Attraction) and ChNPs-HA (Covalently Bonded).

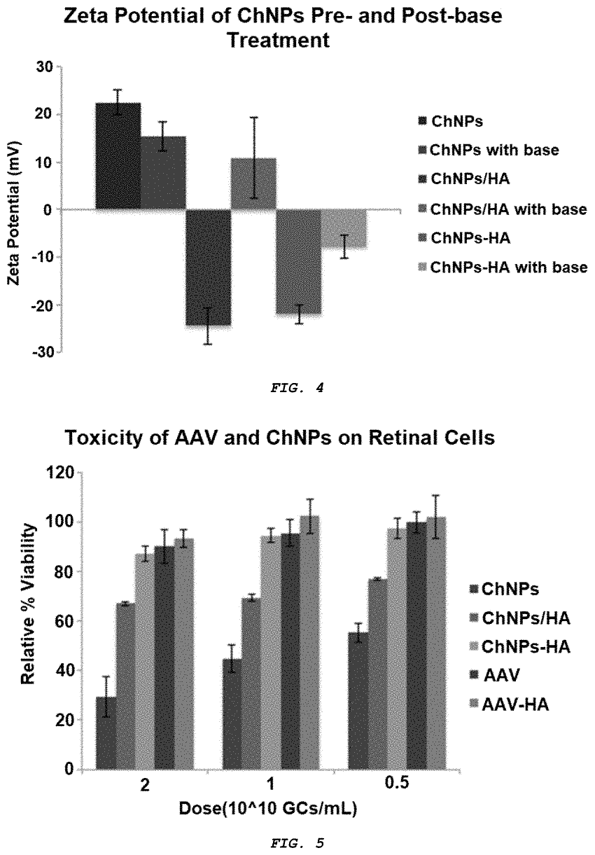

The hydrodynamic size and the zeta potential of ChNPs/HA and ChNPs-HA (0.8×10¹⁰ GC AAV/mL in deionized water) are measured with dynamic light scattering (DLS) using a Malvern Zetasizer Nano ZS (Malvern Instruments, Westborough, MA). HA appeared to be successfully conjugated or electrostatically bound to each sample as demonstrated either by the zeta potential change from positive to negative, or by the size change (in the case of AAV). HA is negatively charged and therefore will have show a negative surface charge, while ChNPs have a positive surface charge and therefore positive zeta. Final solutions were resuspended in DI water and tested with DLS for zeta potential. To remove excess HA, ChNPs with HA were filtered through centrifugal filtration with a size of 100 kDa at 3000 rpm, 30 min at 4° C. for multiple times. After the final wash, the ChNPs with HA were resuspended in 1 mL of H₂O. Charge and size of the ChNPs with HA were measured using Malvern Zetasizer. HA appeared to be successful conjugated or electrostatically bound to each sample as demonstrated either by the zeta potential change from positive to negative (e.g., see FIG. 3B), or by the size change (in the case of AAV) (e.g., see FIG. 3A).

Determining Whether HA is Electrostatically or Covalently Bound to ChNPs.

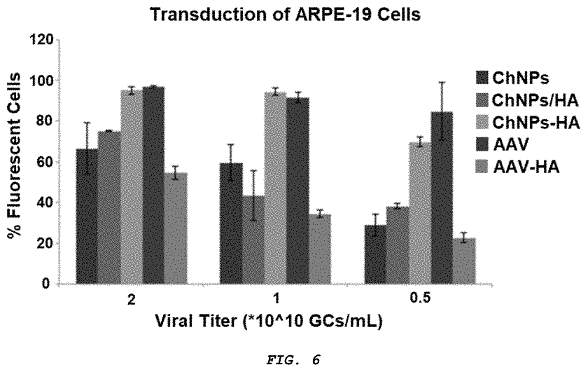

HA is negatively charged and therefore will exhibit a negative surface charge, compared with the ChNPs which will have a positive surface charge and a positive zeta. To ensure that ChNPs-HA and AAV-HA was covalently bound, while ChNPs/HA was electrostatically bound, all groups were incubated in 100 mM sodium hydroxide overnight at room temperature. Sodium hydroxide was removed prior to measurements through a 100 kDa filter centrifugation at 3,000 rpm for 30 minutes at 4° C., and performing multiple washes (×2). A change of zeta potential denotes a loss of HA from the surface of the electrostatically bound ChNPs. ChNPs bound with HA should show negative zeta potential as reflected in the ChNPs-HA covalently bound particles. However, when the electrostatically bound ChNPs/HA were treated with base they now have a positive zeta potential (e.g., see FIG. 4). This denotes a removal of HA from the surface of the ChNPs/HA nanoparticles. The result also confirms that HA was covalently bound in the ChNPs-HA sample.

Assessing the Safety Profile of ChNPs-HA Using Retinal (Arpe-19) Cells.

ARPE-19 cells were seeded at 5,000 cells/well in 100 uL of media and allowed to attach overnight. ChNPs with and without HA, and AAV with and without HA, were delivered to each well in concentrations of 2e10 GCs/mL, 1e10 GCs/mL, and 5e9 GCs/mL and incubated at 37° C. overnight. The following day, media was removed and media-containing 10% MTT was added to the cells. After 4 hours, media and MTT were removed and DMSO was added to each well. The results were read using a plate reader at a wavelength of 570 nm and normalized compared to controls.

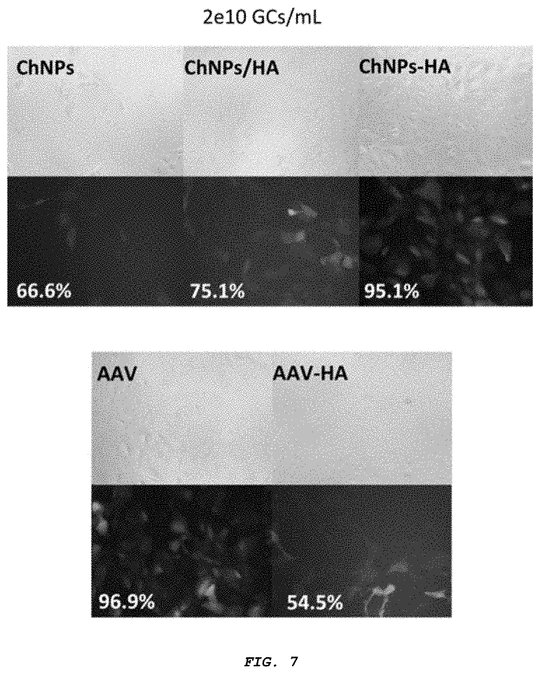

Due to the cationic nature of ChNPs they are more likely to be cytotoxic than other groups. Especially owing to ARPE-19 cells being retinal cells, they will likely be much more sensitive to any degree of ionic density compared with other cells that have been used with ChNPs. The HA coating, however, demonstrated a large reduction in toxicity in ARPE-19 cells for the ChNPs (e.g., see FIG. 5).

Assessing the Transduction Efficiency of ChNPs on Retinal Cells.

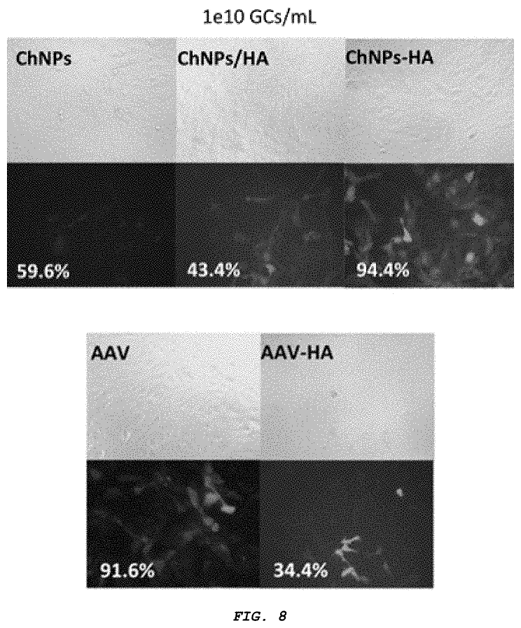

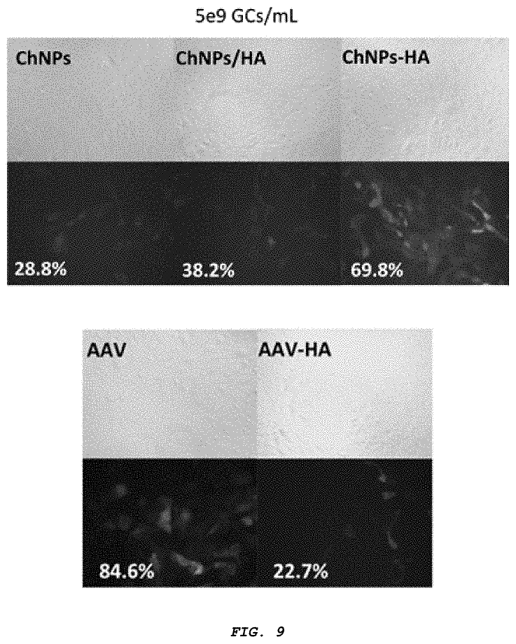

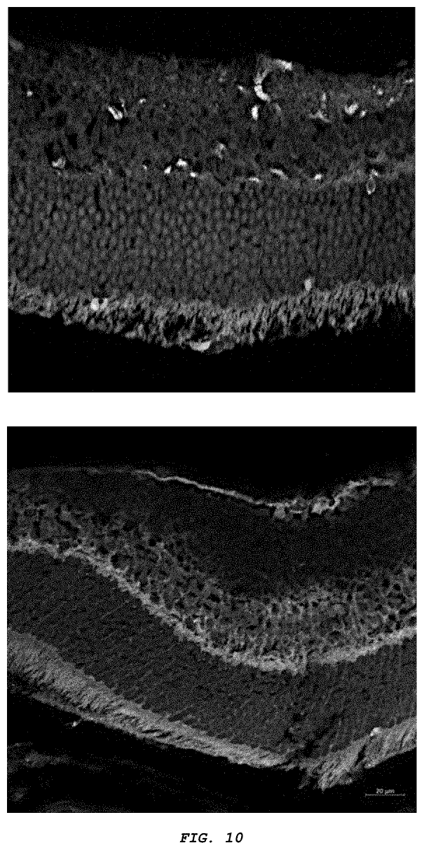

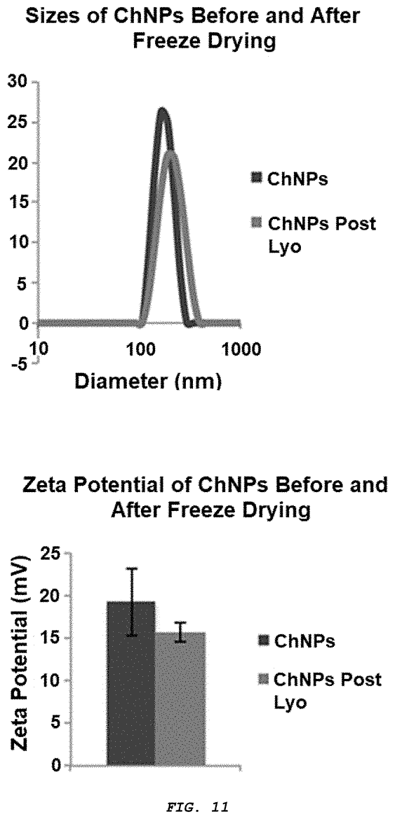

ARPE-19 cells were seeded at 5,000 cells/well in 100 uL of media and allowed to attach overnight. ChNPs with and without HA and AAV with and without HA were delivered to each well, in concentrations of 2e10 GCs/mL, 1e10 GCs/mL, and 5e9 GCs/mL and incubated at 37° C. overnight. The next day media was replaced with fresh media. On days 3, 4, and 5 (post-treatment) fluorescent and brightfield images were recorded and quantified using GUAVA flow cytometry (e.g., see FIGS. 6-9).

ChNPs-HA show vast improvement in transduction of ARPE-19 cells in comparison to uncoated ChNPs. The HA likely binds to CD44 receptors on the surface of the ARPE-19 cells thereby mediating transduction. While ChNPs-HA showed great transduction efficiency, the same could not be said of AAV-HA (e.g., see FIG. 5). This could be for a few reasons. One being that too much HA was conjugated to the surface of the AAV, making it hard for the virus to contact without other mechanisms. More likely, however, AAV-HA exhibits inefficient endosomal escape. The ChNPs will break down in acidic conditions in the endosome and AAV will be released into the cytoplasm, due to the acid-degradable shell. The AAV-HA does not have this built in release mechanism; therefore, it is possible that the AAV has a harder time of breaking out of the HA and cannot efficiently deliver its cargo into the ARPE-19 cells.

It is important to note that ChNPs without HA have artificially inflated transduction efficiency due to the cytotoxic nature of ChNPs. Many of the cells could express auto-fluorescence, or many cells could have been killed, and remaining living cells transduced.

Assessing the Transduction Efficiency of ChNPs on Retinal Cells In Vivo.

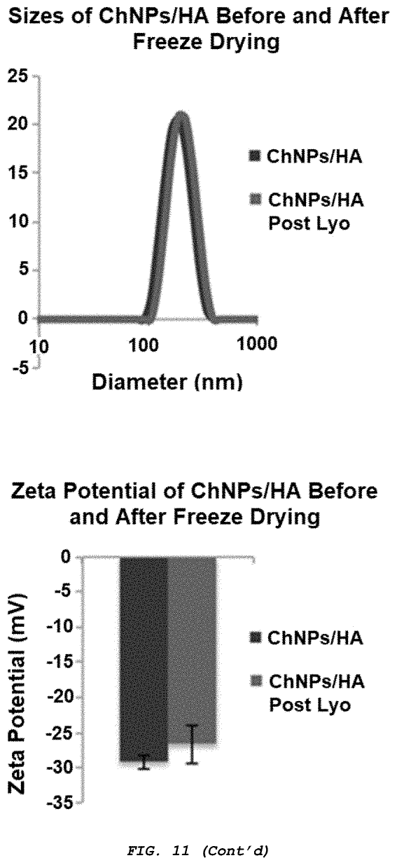

Mice were intravitreally injected with GFP-ChNP or GFP-ChNP-HA. Seven days later, the mice were sacrificed and the mice retinas were sectioned and stained with an Alexa Fluor 633 conjugated anti-GFP antibody (see FIG. 10). It was found that GFP-ChNP-HA localized in the inner retinal cells (see FIG. 10, (lower panel)), while GFP-ChNP did not localize anywhere in particular, and clearly not in the inner retinal cells (see FIG. 10, (upper panel)).

Stability of ChNPs to Freezing and Lyophilization.

For long term storage of the ChNPs in would be advantageous that the ChNPs be freeze-dried and reconstituted but still have meaningful activity. ChNPs were prepared and dispersed in 1 mL of H₂O with 5% glucose (as a cryoprotectant). ChNPs frozen at −80° C. for 4 hours, before being lyophilized overnight. The ChNPs were then resuspended in 1 mL H₂O and tested in ARPE-19 cells, and DLS as described above.

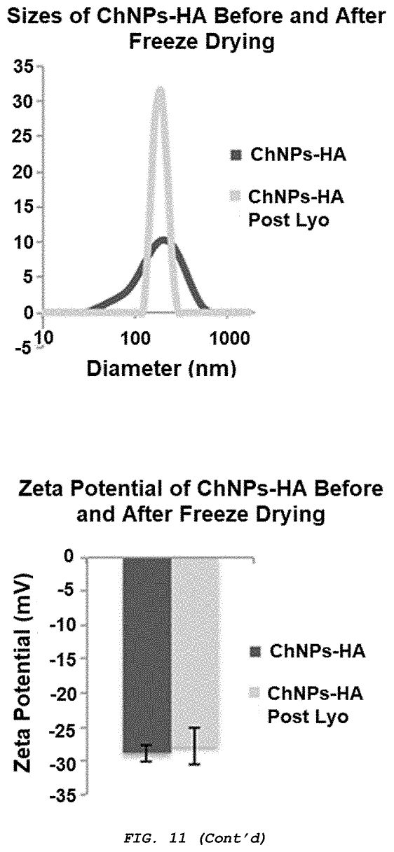

Sizes appeared to be well maintained through freeze drying (e.g., See FIG. 11). Zeta potential values were also within the standard deviations of the original values (e.g., See FIG. 11). Thus, it appears that the structure of the tested particles was maintained even after freeze-drying and reconstituting the particles. It was then important to measure transduction efficiency to ensure activity was maintained as well as structure.

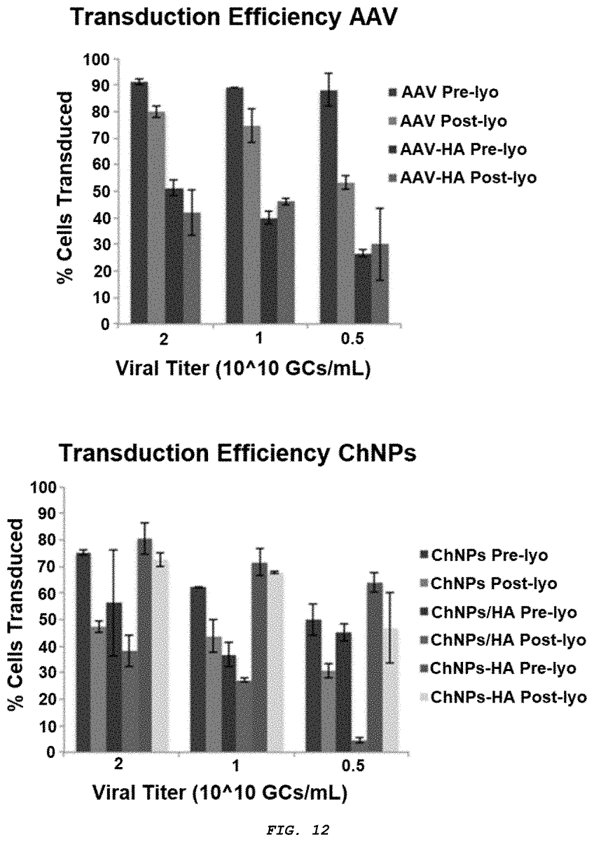

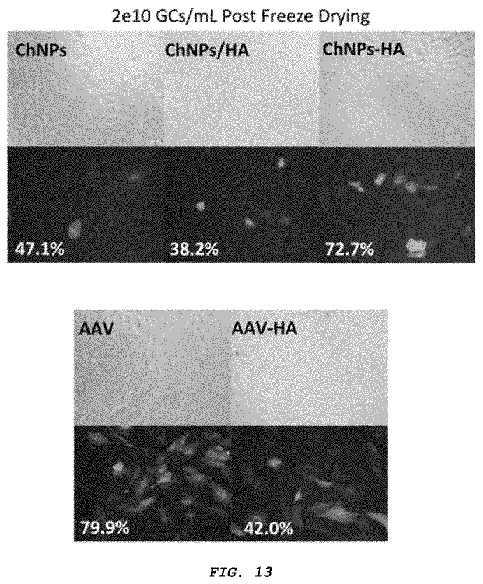

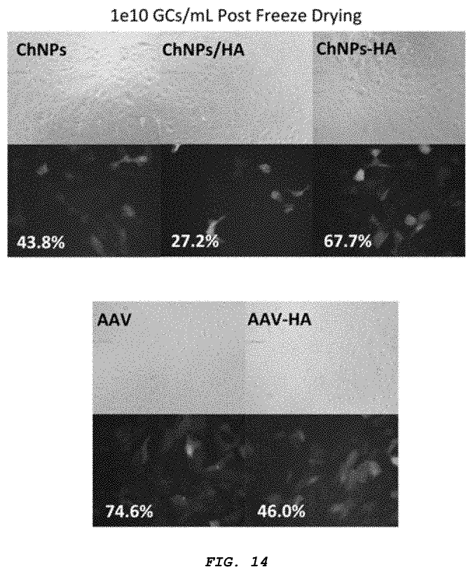

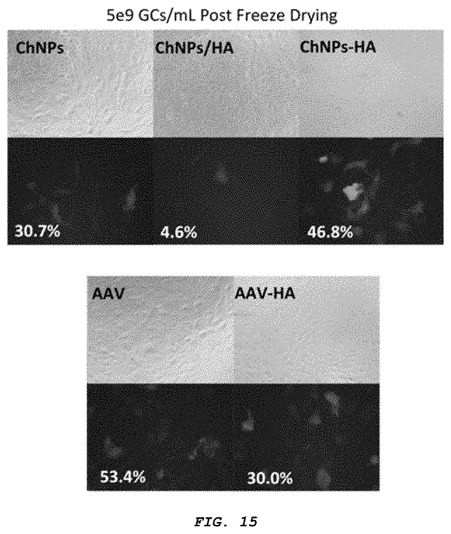

After the freeze-drying cycle, it was readily apparent that ChNPs-HA, AAV, and AAV-HA have the best transduction efficiencies (e.g., see FIGS. 12-15). Thus, the data indicates that ChNPs-HA can withstand freeze-drying processing and still successfully transduce ARPE-19 cells. This suggests that ChNPs exhibit stability in both structure and activity using standard medical storage conditions.

It will be understood that various modifications may be made without departing from the spirit and scope of this disclosure. Accordingly, other embodiments are within the scope of the following claims.

What is claimed is:

1. A hyaluronic acid functionalized chimeric viral/nonviral nanoparticle comprising:
(i) a core comprising a recombinant adeno-associated virus (AAV) that expresses a transgene;
(ii) one or more acid labile degradable polymer layers surrounding the core that may further comprise encapsulated nucleic acids, CRISPR-Cas or CRISPRi systems, therapeutic proteins, or therapeutic drugs, wherein the acid degradable polymer layers hydrolyze in a mildly acidic environment; and
(iii) an outer coating that is in contact with the one or more acid labile degradable polymer layers that is comprised of hyaluronic acid.

2. The hyaluronic acid functionalized chimeric viral/nonviral nanoparticle of claim 1, wherein the core comprises a recombinant AAV that expresses a gene therapy product from a transgene to treat a disease or disorder.

3. The hyaluronic acid functionalized chimeric viral/nonviral nanoparticle of claim 1, wherein the one or more acid labile degradable polymer layers are polyketal-based polymer layers.

4. The hyaluronic acid functionalized chimeric viral/nonviral nanoparticle of claim 1, wherein the hyaluronic acid functionalized chimeric viral/nonviral nanoparticle has a zeta potential from 0 mV to −30 mV.

5. The hyaluronic acid functionalized chimeric viral/nonviral nanoparticle of claim 1, wherein the one or more acid labile degradable polymer layers surrounding the core comprise encapsulated gene silencing/editing oligonucleotides.

6. The hyaluronic acid functionalized chimeric viral/nonviral nanoparticle of claim 5, wherein the gene silencing/editing oligonucleotides are siRNA, miRNA or shRNA.

7. The hyaluronic acid functionalized chimeric viral/nonviral nanoparticle of claim 5, wherein the gene silencing/editing oligonucleotides suppress the expression of a gene whose expression or overexpression is associated with an ocular disease or disorder.

8. The hyaluronic acid functionalized chimeric viral/nonviral nanoparticle of claim 7, wherein the gene silencing/editing oligonucleotides suppress the expression of the IL-1β, TNFα, COX-2, HIF-1α, VEGF-A, VEGF-B, PIGF, VEGFR1, VEGFR2, FGF-b, A-RAF, mTOR, MMM-2, MMP-9, and/or Integrin avb3 gene.

9. The hyaluronic acid functionalized chimeric viral/nonviral nanoparticle of claim 5, wherein the gene silencing/editing oligonucleotides suppress the expression of a gene whose expression or overexpression is associated with a liver disease or disorder.

10. The hyaluronic acid functionalized chimeric viral/nonviral nanoparticle of claim 9, wherein the gene silencing/editing oligonucleotides suppress the expression of mutant alleles associated with a liver disorder, LDL receptors, ApoB-100, proprotein convertase subtilisin/kexin type 9 (PCSK9), Fas-mediated apoptosis proteins, and miRNAs associated with hepatic lipid metabolism.

11. The hyaluronic acid functionalized chimeric viral/nonviral nanoparticle of claim 1, wherein the outer coating comprising hyaluronic acid is contacted with the one or more acid labile degradable polymer layers through electrostatic interactions.

12. The hyaluronic acid functionalized chimeric viral/nonviral nanoparticle of any one of claim 1, wherein the outer coating comprising hyaluronic acid is contacted with the one or more acid labile degradable polymer layers through covalent bonds.

13. A pharmaceutical composition comprising the hyaluronic acid functionalized chimeric viral/nonviral nanoparticle of claim 1 and a pharmaceutically acceptable carrier.

14. The pharmaceutical composition of claim 13, wherein the pharmaceutical composition is formulated for administration by intravitreal injection, parenterally, or by subretinal injection.

15. A method of treating a subject that has an ocular disease or disorder, comprising:
administering to the subject an effective amount of the hyaluronic acid functionalized chimeric viral/nonviral nanoparticle of claim 1.

16. The method of claim 15, wherein the ocular disease or disorder is selected from the group consisting of age-related macular degeneration, retinitis pigmentosa, Stargardt disease, Usher syndrome, rod-cone dystrophy, Bardet-Biedl syndrome, diabetic retinopathy, choroideremia, Oguchi disease, malattia leventinese, intraocular cancer, retinoblastoma, central retinal vein occlusion, branched retinal vein occlusion, blue-cone monochromacy, albinism, bacterial keratitis, chorioretinopathy, glaucoma, conjunctivitis, cytomegalovirus retinitis, drusen, Fuchs' dystrophy, fungal keratitis, viral keratitis, macular telangiectasia, optical neuritis, and scleritis.

17. The method of claim 15, wherein the ocular disease or disorder is age-related macular degeneration.

18. The method of any one of claim 15, wherein the hyaluronic acid functionalized chimeric viral/nonviral nanoparticle is administered in combination with an ophthalmological or eye treatment.

19. A method of treating a subject that has a liver disease or disorder, comprising:
administering to the subject an effective amount of the hyaluronic acid functionalized chimeric viral/nonviral nanoparticle of claim 1.

20. The method of claim 19, wherein the liver disease or disorder is selected from the group consisting of hepatitis A, hepatitis B, hepatitis C, fatty liver disease, liver cancer, Wilson disease, hemochromatosis, Alagille syndrome, alcohol-related liver disease, alpha-1 antitrypsin deficiency, autoimmune hepatitis, biliary atresia, cirrhosis, Crigler-Najjar Syndrome, Galactosemia, Gilbert Syndrome, hepatic encephalopathy, hepatorenal syndrome, lysosomal acid lipase deficiency, non-alcoholic fatty liver disease, non-alcoholic steatohepatitis, primary biliary cholangitis, primary sclerosing cholangitis, Reye syndrome, Type I glycogen storage disease, hemophilia A and hemophilia B.
